(19) United States
(12) Patent Application Publication
(10) Pub. No.: US 2016/0022402 A1
(43) Pub. Date: Jan. 28, 2016

(54) METHOD AND APPARATUS FOR SELF-ADHESIVE MEDICAL MESH DEPLOYMENT

(71) Applicant: Houman Soloman, Hermosa Beach, CA (US)

(72) Inventor: Houman Soloman, Hermosa Beach, CA (US)

(21) Appl. No.: 14/747,625

(22) Filed: Jun. 23, 2015

Related U.S. Application Data
(60) Provisional application No. 61/998,771, filed on Jul. 3, 2014.
(60) Provisional application No. 62/055,956, filed on Sep. 26, 2014.

(51) Int. Cl. A61F 2/00

(52) U.S. Cl. CPC A61F 2/0063; A61F 2002/009; A61F 2210/0076; A61F 2220/0016

(57) ABSTRACT

Mesh for laparoscopic surgery includes, in addition to self-attaching mesh, a carrier or control layer used during insertion and alignment to reduce unwanted self-attachment to tissue in or outside of the target area to be repaired. The carrier has a control surface to which the self-attaching surface of the mesh does not attach and a tissue contact surface which has limited if any attachment to tissue. The mesh and carrier may be combined to form a layered mesh insertable during limited access surgery after which the carrier may be removed by trocar. The carrier may allow a limited portion of the working mesh surface to contact tissue in the cavity and thereby improve manipulation of the mesh within the cavity and re-alignable, pre-alignment of the mesh with the target area. The carrier includes tabs for maintaining alignment with the mesh in the cavity and for gripping to realign or remove the carrier from the cavity. The carrier may be in the form of paper, plastic and/or in an envelope configuration.

### RELATED APPLICATIONS

This application claims the priority of the filing date of U.S. Provisional Applications Ser. No. 61/998,771 filed Jul. 3, 2014 and Ser. No. 62/055,956 filed Sep. 26, 2014.

### FIELD OF THE INVENTIONS

This invention is related to the use of self-adhesive medical mesh in minimally-invasive surgery, such as endoscopy and particularly laparoscopy, within the patient and in particular to techniques for improving the deployment and use of self-adhesive medical mesh used in such operations.

### BACKGROUND OF THE INVENTIONS

Self-attaching medical mesh, such as self-adhesive or self-fixating mesh is used in operations, for example, for repairing hernias. For minimally invasive surgery, self-adhesive medical mesh has been developed which is designed to include a working surface which attaches or otherwise adheres to organic surfaces such as tissues in the human body and is thereby often used to provide a surface which a) adheres to a wound or other damaged organic surface and b) provides a surface on which organic material will naturally grow to reinforce or otherwise improve the damaged organic surface. Many other uses of self-adhesive medical mesh are possible and/or are known, such as to provide support for a currently improperly supported organs and many other uses are expected to be developed.

For example, in a conventional, non-invasive operation to repair a hernia with mesh, the self-adhesive medical mesh is typically inserted, for example via trocar through the patient's skin, into the vicinity of the damaged organic surface to be repaired. The self-adhesive medical mesh used is typically sized to cover the area of the hernia and preferably to extend beyond the hernia to adhere to undamaged organic material to provide more secure support for the mesh while the body repairs the hernia and also to support the buildup of organic material on and around the mesh once the damaged area is protected by this build up.

Such self-attaching mesh is typically configured when rolled up, or otherwise furled, to fit within the trocar for insertion by the surgeon into the body cavity in the vicinity of the hernia. Once inserted, the mesh is then typically unfurled by the surgeon and placed where needed, such as in contact with hernia itself and surrounding undamaged tissue. Thereafter, in a successful operation, the mesh serves as an architectural support natural to repair the hernia and natural buildup of organic material over time on the self-adhesive medical mesh serves further serves to strengthen and protect such damaged areas.

A wide variety of medical uses for self-adhesive medical mesh and similar materials, if any, which can be non-invasively inserted or otherwise deployed in the body to adhere to damaged and/or undamaged organic surfaces to repair and support internal damaged surfaces or support organs or the like, are possible. However, such medical uses typically require intensive labor from a skilled surgeon to unfurl the mesh, if necessary, properly position the self-adhesive mesh where required and urge the mesh into contact with the body where appropriate.

One of the problems with conventional techniques is that upon insertion in the body, while unfurling and/or during placement, the adhesive qualities of the self-adhesive mesh which are necessary for its ultimate use unfortunately cause the mesh to bond with any organic surface contacted. The surgeon must therefore touch, unfurl and/or move the mesh to avoid and/or undo this unwanted bonding until final placement is achieved.

What is needed are techniques for reducing the unwanted mesh bonding and thereby reduce the level of surgeon labor and skill required for such operations in order reduce the time required for such operations and make them more widely available and at lower costs.

### SUMMARY

The objects of this invention are achieved, in general, by reducing the level of adhesion of at least some part of at least one surface of the self-adhesive medical mesh during and/or before the operation without reducing the long term efficacy of the mesh to adhere to organic surfaces in the body and provide support for organic material formed by the body thereon.

A sheet of material may be applied as a protective layer to at least a portion of one surface of the self-adhesive medical mesh to temporarily reduce or eliminate adhesion of that mesh surface to organic materials during at least a portion of the operation. Once the mesh is inserted and unfurled within the body, if necessary, and properly positioned where needed by the surgeon, the adhesion of that surface to the organic surfaces in the body may be wholly or partially restored either during the operation by the surgeon and/or in part thereafter for example by dissolution of the sheet and/or otherwise restoring the efficacy of mesh to adhere to organic materials.

In a first embodiment, an adhesion reducing protective layer is affixed to at least a portion of one surface of the self-adhesive medical mesh to provide a multilayered, controlled adhesion medical mesh. The multilayered controlled adhesion mesh is then folded and/or furled so that in can be inserted by the surgeon through a trocar into the vicinity of the area to be repaired. Once the controlled adhesion self-adhesive medical mesh has been inserted within the body, it is manually unfolded or unfurled, positioned by the surgeon as desired and the adhesion reducing protective layer is removed from the mesh preferably while held in position manually by the surgeon using a grabber preferably inserted through the trocar after the layered mesh was deployed.

The protective layer may, for example, be provided with one or more strings or other devices which can be controlled by a first grabber while a second grabber is used to maintain the desired position of the multilayered mesh. One or more of the strings may then be operated by the first grabber to split the protective layer into multiple subparts. Preferably the subparts may then be removed from the body via the trocar using other ones of the strings.

For example, the strings may be color or otherwise coded. Some strings may be associated with perforations or other intentional weaknesses in the protective layer to that the layer, when appropriate may be split into multiple pieces. The same or other strings may be positioned outside of the body through the trocar before the mesh was inserted or by operation of a grabber. Such strings may then be used by the surgeon to remove unwanted portions of the protective layer via the trocar.

In other preferred embodiments, the protective sheet may have a physical memory so that a furled sheet may automatically unfurl once inside the body, perhaps after removal of a suture or other mechanism for maintaining the mesh rolled up for insertion via the trocar.

Self-attaching layered mesh for endoscopic, laparoscopic and other limited access surgery is disclosed which has medical mesh with a working surface which self-attaches to tissue; and an adhesion control layer having a tissue contact surface which does not self-attach to tissue and a control surface to which the working surface does not self-attach. The medical mesh and control layer form a layered mesh with the working surface in contact with the control surface which is sized for insertion during controlled access surgery via a trocar into a cavity.

The layered mesh control layer may be configured to selectively self-attach to tissue in a target area to be repaired during a preliminary alignment of the medical mesh with said tissue and fully attaching thereto after removal of the control layer from the cavity via the trocar. An alignment area of the working surface may be unblocked by the control layer for more convenient positioning of the layered mesh in a desired alignment with the tissue to be repaired which is not hindered by self-attachment of other areas of the working surface to tissue inconsistent with the desired alignment.

An alignment control area of the control layer, for example one or more alignment tabs, may be provided to control alignment of the control surface with the working surface. The alignment control area may include an alignment portion of the control surface which attaches to a corresponding portion of the working surface with a lower strength of attachment than the strength of attachment between the alignment area of the working surface and the tissue so that after the layered mesh is positioned in the desired alignment with the tissue to be repaired, the control layer may be separated from the working surface for removal via trocar without loss of the desired alignment between the working surface and the tissue to be repaired. A plurality of alignment control areas and the strength of attachment of the plurality of alignment control areas to the tissue to be repaired is less than the strength of attachment between the alignment area of the working surface and the tissue.

The alignment control area may include an extension of the control layer extending across a first edge of the working surface to the non-working surface forming a barrier against movement of the mesh within the layered mesh in at least one direction. A plurality of extensions of the control layer extending across a plurality of edges of the working surface to the non-working surface may form a barrier against movement of the mesh in a plurality of directions.

The alignment area of the working surface may be adjacent an alignment edge of the working surface and have a plurality of extensions of the control layer extending across a plurality of edges of the working surface to the non-working surface forming a barrier against movement of the mesh in a plurality of directions, at least one of the plurality of extensions extending across the alignment edge. One of the extensions may include a tab in contact with the non-working surface of the mesh easily removed from the non-working surface during surgery to aid in peeling the alignment area of the working surface from tissue in the cavity during realignment of the layered mesh. A removal tab adjacent an edge of the control layer opposite the alignment area may be liftable from the non-working surface during surgery to aid in removing the control layer from the cavity.

An extension of the control layer across the non-working surface to a second edge thereof may form a barrier against movement of the mesh within the layered mesh in the direction of the first and in the direction of the second edge. The control layer may not form a barrier against movement of the mesh within the layered mesh in the direction of a third edge of the mesh and a removal tab, grippable by an instrument in the cavity, may be provided for removal of the control layer from the layered mesh in the cavity in the direction of the third edge.

The control layer further may include lines of reinforcement material resisting tearing forces applied to the control layer during endoscopic removal of the control layer.

The mesh may be a woven surgical material, one surface of which is configured as the working surface, the control layer may be a second mesh of surgical material woven substantially more tightly than the mesh to reduce or eliminate self-attachment thereto by the working surface. The control layer may include a layer of non-woven surgical material applied to one surface of the second mesh to further reduce or to eliminate self-attachment thereto by the working surface.

In another embodiment, an adhesion control carrier is disclosed for use with self attaching medical mesh in laparoscopic and other limited access surgery, the adhesion control layer having a tissue contact surface which does not self-attach to tissue and a control surface to which a self-attaching surface of the medical mesh does not self-attach, the control layer configured to selectively self-attach to tissue in a target area to be repaired during a preliminary alignment of the medical mesh with said tissue and fully attaching thereto after removal of the control layer from the cavity via the trocar.

### BRIEF DESCRIPTION OF THE DRAWINGS

FIG. 1 is an isometric exploded view of controlled adhesion, layered self-adhesive medical mesh 10 including self-adhesive medical mesh 12 and adhesion controlling layer 14 generally positioned in situ above target area 16 including damaged organic tissue 18, such as a hernia, within and surrounded by undamaged organic tissue 20 of target area 16.

FIG. 2 is an unexploded isometric view of controlled adhesion, layered self-adhesive medical mesh 10 in which mesh layer 12 is partially cutaway as illustrated by dotted lines. Mechanical connecting clip 26 is illustrated to shown how portion 25 of mesh layer 12 may be temporarily held against adhesion control surface 22 of control layer 14 to form controlled adhesion self-adhesive medical mesh 10 for placement, for example, against hernia 18.

FIG. 3 is an isometric view of controlled adhesion self-adhesive medical mesh 10, in which in which mesh layer 12 is not shown and control layer 14 is shown as partially transparent, for clarity, and lower or tissue contact surface 24 of layer 14 is positioned in contact with target area 16. Tear string 30 is illustrated positioned within tear string separation zone 32 while upper removal string 38 is attached to upper piece 34 for removal and lower removal string 40 is attached to lower piece 36 prior to removal of control layer 14.

FIG. 4 is a top view of control layer 14, held in position with mesh 12 (not shown in this figure) by a plurality of control clips 26, and illustrating the locations of tear string 30 in separation zone 32, removal strings 38 and 40 and the placement of grabber tips 28 and 29 in upper and lower pieces 34 and 35 of layer 14.

FIG. 5 is a top view of control layer 14, as a partial alternate to the embodiment shown in FIG. 4, illustrating the use of notch 44 in layer 14 which may be used by the pressure of grabber tip 28 on a portion of target area 14 exposed by notch 44 to selectively hold mesh 12 (not shown in this figure) in position with regard to target area 16 during alignment and while control layer 14 may be removed from between mesh 12 and target area 16, for example by operation of short removal string 46 within the body and/or in combination with operation of long removal string 48 which may be positioned outside the body.

FIG. 6 illustrates the use of roll clamp 54, such as a suture, to hold controlled adhesion self-adhesive medical mesh 10 in a compact form with control layer 14 on the outer surface of roll 52 for more convenient insertion into the body via a trocar or similar device (not shown in the this figure).

FIG. 7 is a top view of mesh 10 in an unfurled or flattened configuration in alignment of a top view of mesh 10 when formed into roll 52 together with their various dimensions.

FIG. 8 is a slightly exploded top view of a pair of rolls 52, separated by endcap 56, for insertion together into the body via trocar or similar surgical tool.

FIG. 9 is a top view of a foldable embodiment version of mesh 10, when unfurled, including left, center and right panels.

FIG. 10 is a cross sectional view of foldable mesh 10 when the right and left panels are folded onto the central panel.

FIG. 11 is a top view of foldable mesh 10 of FIG. 10 further folded along center line 60 of the central panel ready for insertion into the body via a surgical tool such as a trocar, not shown in this figure.

FIG. 12 is a cross sectional view of foldable mesh 10 positioned on target area 16 in the body.

FIG. 13 is a cross section view of foldable mesh 10 positioned on target area 16 after the left and right panels of control layer 14 have been removed.

FIG. 14 is a cross sectional view of self-adhesive medical mesh 12 showing full deployment on target area 16 after the center panel of control layer 14 has been removed.

FIG. 15 is more exploded view of FIG. 1 in which controlled adhesion self-adhesive medical mesh 10 is exploded and shown above target area 16 for clarity in description of control and lower surface layers 22 and 24 on control layer 14.

FIG. 16 is a cross sectional view of an alternate to the embodiment of FIG. 11.

FIG. 17 is a cross sectional view of an alternate to the embodiment of FIG. 12.

FIG. 18 is an isometric view of the underside or tissue contact surface 24 of control layer 14 showing notch 44, realignment tabs 72, holding tabs 74, stress reducing reinforcement lines 76, tab folding arrow 78, patch 80, alignment point 82 and removal strings 48.

FIG. 19 is a cross sectional illustration of self-adhesive or self-fixating medical mesh 12 having a hook like mechanism for adhesion and three embodiments of adhesion control layers or carriers.

FIG. 20 is an illustration of an open polyester mesh, including self-fixating hook like mechanisms on strings within the mesh, in contact with control surface 92 of carrier 90.

FIG. 21 is an isometric view of controlled adhesion, layered mesh 10 including self-adhesive medical mesh 12, control layer 14 underneath self-adhesive medical mesh 12 and visible only as tabs 74 and 72 (folded over self-adhesive medical mesh 12), positioned over target area 16 for alignment of layered mesh 10 over hernia 18 and undamaged tissue 20.

FIG. 22 is an isometric view of layered mesh 10 including folded removal tab 75.

FIG. 23 is an exploded isometric view of layered mesh 10 including coarsely woven polyester mesh forming medical mesh 12 and control layer surface 22 of control layer 14 in alignment above target area 16.

FIGS. 24 and 25 are illustrations of a conventional medical mesh 112 in an outline and a folded view for convenience in describing the embodiments of FIGS. 26 and 27.

FIGS. 26 and 27 are illustrations of an embodiment in which control layer 14 is in the general form of an envelope shown as envelope carrier 114.

### DETAILED DESCRIPTION OF THE INVENTIONS

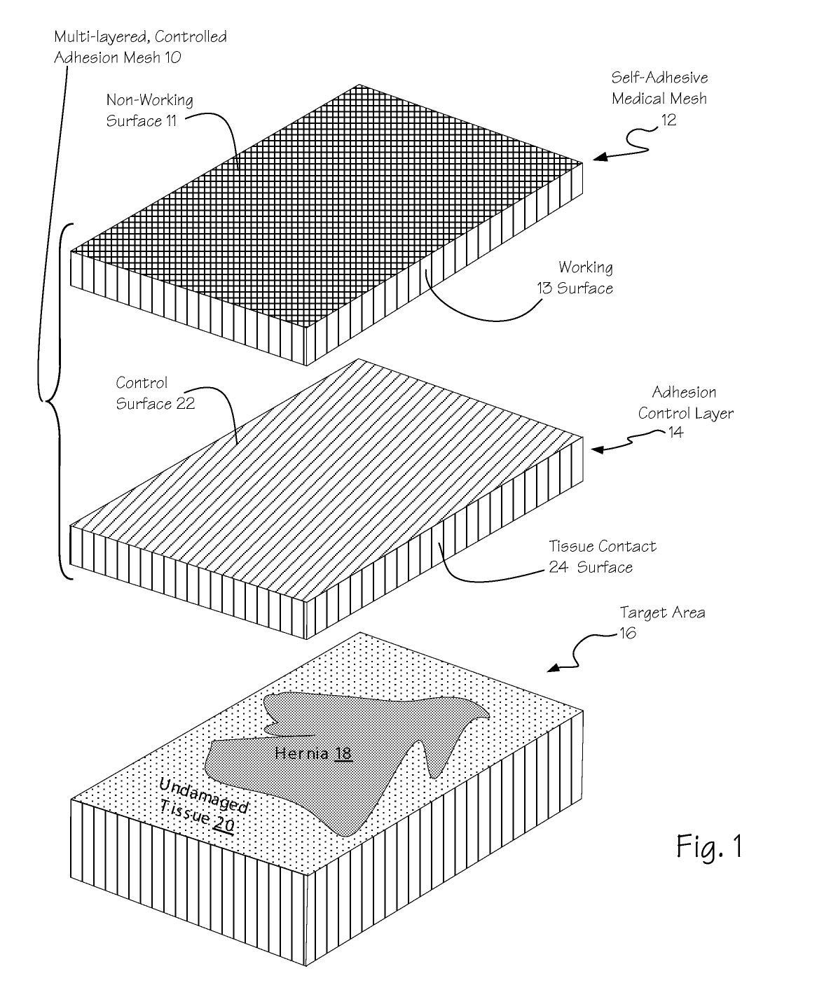

Referring now to FIG. 1, controlled adhesion, layered self-adhesive medical mesh 10, including self-adhesive medical mesh 12 and adhesion controlling layer 14, are generally positioned during surgery in situ above target area 16 where damaged organic tissue 18, such as a hernia, are included within and surrounded by undamaged organic tissue 20 of target area 16. This figure is intended to establish the relative orientation of the elements shown. The relative sizes and thicknesses portrayed in this figure are for convenience and not intended to reflect the relative sizes of the components shown in this figure. In other operations, the relative layered orientation of mesh 12, layer 14 and target area 16 will remain the same even if target area 16, for example, is related to one or more mounting areas related to the support of an organ, or the like, not shown.

Now in greater detail, self-adhesive medical mesh 12 may be a medical mesh used in medical operations, such as hernia repair, and in fact may be a woven or other mesh made of materials which easily bond to organic tissue within the human body during and/or resulting from placement therein by a surgeon. Such mesh may be available, for example from Covidien (Dublin, Ireland) under the brand name ProGrip.

Control layer 14 made be made of any suitable material to be positioned in contact with the surface of self-adhesive medical mesh 12 which will be positioned on target area 16 including hernia 18. The qualities required for the materials of control layer 14, in addition to the obvious need for materials which can safely be used within human bodies during operations, relate to the relative strengths of adhesive or attachment or other connection between a) control surface 22 of control layer 14 with working 13 surface of self-adhesive medical mesh 12 above it and b) lower or tissue contact surface 24 of control layer 14 and the surface of target area 16.

In particular, during the medical operation, working surface 13 of self-adhesive or self-attaching medical mesh 12, may be held against upper surface 22 of control layer 14 by mechanical means such as clips not shown in this figure and tissue contact surface 24 of control layer 14 may have little or no adhesion with tissue, e.g. tissue in target area 16. As a result, in use, the surgeon may advantageously deploy layered, controlled adhesion mesh 10 and orient mesh 10 into the proper position with regard to target area 16 by pushing layered mesh 10 into contact with undamaged tissue 20 and sliding layered mesh 10 along the surface of tissue 10 until properly aligned with hernia 18.

It is important to note that reduced friction between tissue contact surface 24 and tissue encountered during the operation, e.g. in target area 16, such as undamaged tissue 20 and hernia 18, substantially reduces the difficulty and time required for even a less experienced surgeon to properly align self-adhesive medical mesh 12 with target area 16. The next step after alignment, of course, is to remove control layer 14 from between self-adhesive medical mesh layer 12 and target area 16 and from the body cavity.

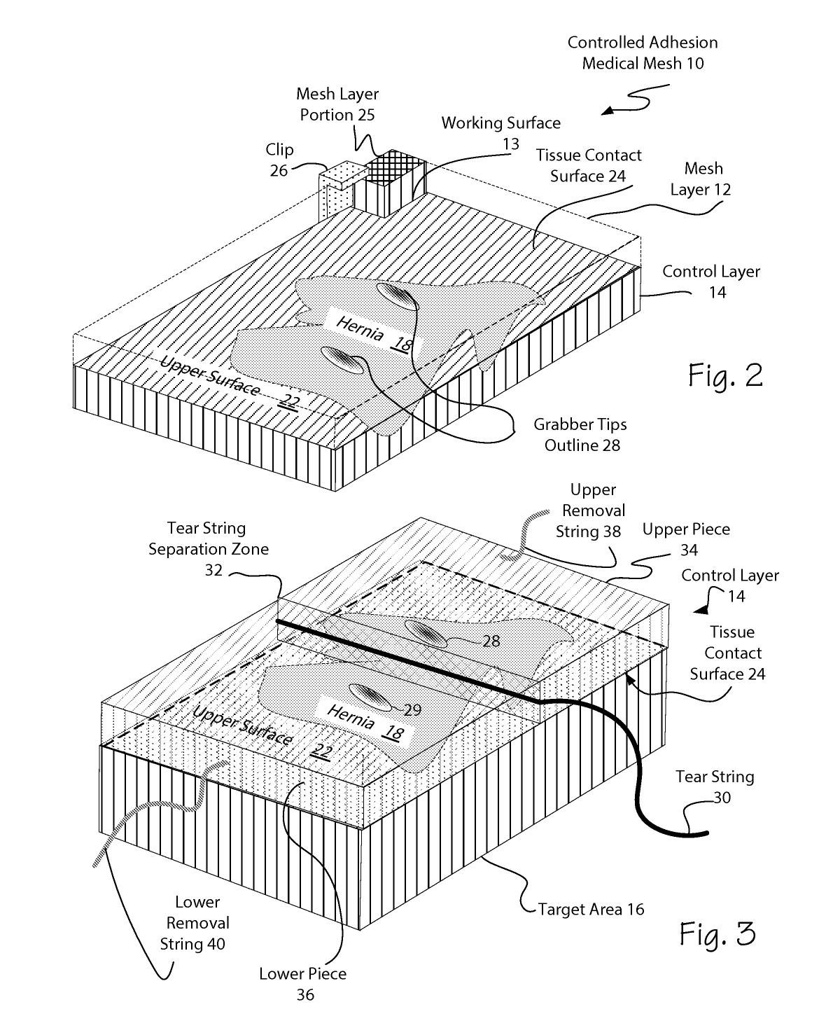

Referring now to FIG. 2, controlled adhesion self-adhesive medical mesh 10 is shown in an unexploded view with most of self-adhesive medical mesh layer 12 cutaway leaving mesh layer portion 25 visible in an upper corner. A plurality of mechanical temporary connecting devices, such as clips 26, a portion of one of which is shown adjacent mesh layer portion 25 to illustrate how such clips could hold mesh layer 12 and control layer 14 together so that. Once layered mesh 10 is accurately positioned over the area to be repaired, as indicated by hernia 18, within in target area 16, clips 26 may be removed by grabber or other convenient means. Layered mesh 10 may be held in place by pressure of tips 28 of a grabber positioned over some portion of target area 16, illustrated in FIG. 1. An approximate position of tips 28 and 29 may be over hernia 28 as illustrated by grabber tips outlines 28 superimposed on the outline of hernia 18. Thereafter control layer 14 may be removed by the surgeon in order to place mesh layer 12 in direct contact with target area 16 including hernia 18.

Referring now to FIG. 3, in an unexploded view, mesh layer 12 of layered mesh 10 has been removed for clarity showing control layer 14 positioned on target area 16. Control layer 14 is shown as semitransparent in order to illustrate the relative position of hernia 18 of target area 16 together with grabber tip pressure positions 28 and 29. Tear string 30 may be a color or other coded string extending back through the trocar to the surgeon for operation, and/or may be available for operation within the body by grabber.

While pressure by grabber is maintained for example on opposite sides of separation zone 32 associated with tear string, as illustrated by the outlines of tips 28, operation of tear string 30 may be used to cause control layer 14 to be divided or separated into two pieces, upper piece 34 and lower piece 36, along tear string separation zone 32.

Once the upper and lower pieces of control layer 14 have been separated, control layer upper piece 34 may be removed by maintaining the position of lower piece 36 (and therefore the portion of mesh layer 12 there above not shown in this figure) with respect to hernia 18 by removing pressure at grabber tip 28 while retaining pressure at grabber tip 29. Operation of upper removal pull string 38 may then be used to pull upper piece 34 out from under mesh layer 12 and out of the body via the trocar without disturbing the alignment of mesh layer 12 to target area 16. Reintroducing pressure via grabber tip 28 will then bond an appropriate portion of mesh layer 12 to the portion related portion of target area 16.

Thereafter, operation of lower removal string 40, grabber tips 28 and 29 and lower portion removal string 40 in a similar manner may be used to remove lower portion 36 so that the entirety of mesh layer 12 may be bonded and adhered to target area 16 to complete the operation.

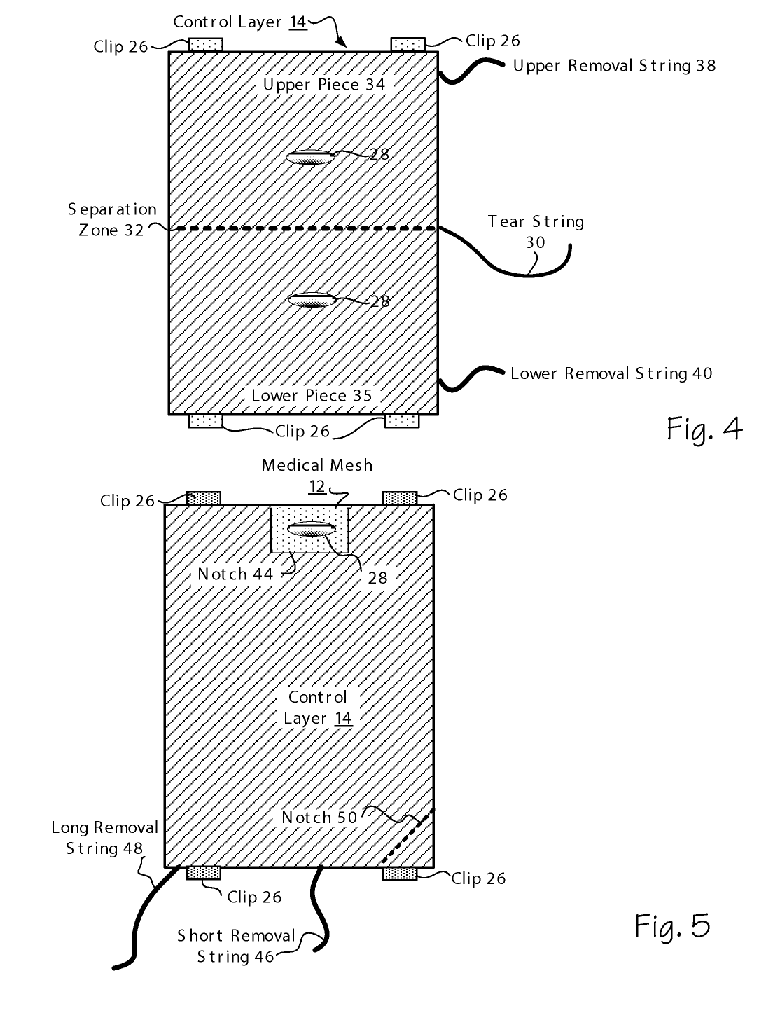

Referring now also to FIG. 4, a top view of controlled adhesion layered mesh 10 is shown with self-adhesive medical mesh 12 removed, as removed, as in FIGS. 2 and 3, to make control layer 14 visible. Four clips 26 for holding self-adhesive medical mesh 12 to control layer 14 are shown which are used to combine these layers in order to form layered mesh 10. Grabber pressure points 28 are illustrated above and below separation zone. As can be seen in this view, and in the same manner described above with respect to FIG. 3, operation of tear string 30 by the surgeon causes upper piece 34 of control layer 14 to become separated from lower piece 35 along separation zone 32. Thereafter, one of upper string 38 or lower string 40 may be pulled by the surgeon, by a grabber within the body or more directly by the surgeon if the strings extend through a trocar outside of the body.

When one of these strings is pulled, the related grabber pressure point 28 is substantially reduced or eliminated so that the related upper or lower piece of control layer 14 can be at least partially removed from layered mesh 10. Thereafter, the removed pressure point 28 can then be reapplied to push self-adhesive medical mesh 12 into a bonding contact with the related portion of target area, not shown in this figure. Thereafter, the removal string can be pulled to more completely removed the related upper or lower piece of control layer 14 from layered mesh 10. The same string can then or later be used to remove the piece of control layer 14 from the body.

After the first such removal string 38 or 40 has been pulled to remove a portion of control layer 14, the operation may be repeated by pulling the other such string to at least partially (or fully) remove the other portion of control layer 14 from between mesh 12 and target 16 so that at least a partial bonding between these surfaces can be made. A grabber may preferably be used to apply additional pressure between mesh 12 and target area 16 where already at least partially bonded in order to maintain alignment of mesh 12 and target area 16 while the other portion of layer 14 is removed.

Referring now to FIG. 5, a bottom view of layered mesh 10 is shown to illustrate an alternate technique for maintaining alignment during deployment. A cross sectional view of plurality of clips 26 are shown which are used to maintain self-adhesive medical mesh 12 in alignment with control layer 14 to form layered mesh 10. In this bottom view, a portion of self-adhesive medical mesh 12 is visible in cutout or notch 44 of control layer 14.

In this technique, a preliminary alignment of layered mesh 10 over target area 16 may be maintained at least in part by the adhesion of the portion of self-adhesive medical mesh 12 with a portion of target 16 (not shown in this figure) available for contact there between through notch 44 of control layer 14. In particular, although a portion of mesh 12 may available for selective attachment with target area 16 while the surgeon is making a preliminary alignment, the surface area contact available for adhesion during alignment may be advantageous limited because of the relative smaller are of notch 44. There are several benefits as a result of this selective attachment, that is, the reduced area of the working surface available for self attachment to tissue during alignment when compared with the substantially greater are for self-attachment after permanent alignment, i.e., after the surgery when control layer 14 has been removed.

Conventionally the medical mesh has been crumpled, rolled and folded in order to fit within a trocar for insertion in an existing or surgically created body cavity. The working surface of such mesh as it is being unfurled often self-attaches to tissue not intended to be attached to the mesh, for example, tissue outside of the target area such as tissue dislodged while forming or clearing the body cavity while preparing for mesh insertion. This attachment to such tissue typically has to be removed before the surgeon is able to align the mesh with the target area to be repaired.

Similarly, during alignment, the self-attaching nature of the mesh working surface often causes the medical mesh to self-attach to tissue of the target area in an improper or unintended alignment. Again, such unintended attachments may have to be removed before a proper alignment may be achieve and the mesh permanently attached as desired for the surgery.

By presenting a reduced area of working surface 13 for self-attachment, i.e., the area within notch 44, less unintended attachment occurs with either tissue outside of target area 16 and/or with tissue in target area 16 in an undesirable alignment. Further, the surgeon may do a preliminary alignment of only the working surface area exposed via notch 44 and if necessary more easily realign layered mesh 10 if necessary because of the reduced area of self attaching working surface 13 which is attached to tissue before control layer 14 is removed. Advantageously, the use of notch 44 may not reduce the total surface area of self attaching working surface 13 which becomes self-attached to target area 16 after removal of adhesion control layer 14.

After the surgeon has made the preliminary alignment, grabber pressure point 28 on the portion of self-adhesive medical mesh visible in notch 44 may be used to enhance the adhesion available of layered mesh 10 with target area 16 sufficiently to perform other tasks. One advantage of this technique is that further alignment corrections can be more easily made even after adhesion only via notch 44 because that level of adhesion is substantially less than the adhesion the entire surface of self-adhesive medical mesh 12 to target area 16.

Until final alignment of layered mesh 10 with target 16 is achieved, layered mesh 10 may be conveniently be removed and/or realigned with target 16 because of the limited adhesion if any, between the small portion of self-adhesive medical mesh 12 available for contact with target area 16 when urged, for example, by pressure of grabber tip 28 on self-adhesive medical mesh 12. Once final alignment is achieved, control layer 14 may conveniently be removed by pulling either short removal string 46 with a grabber inside the body and/or by pulling long removal string 48 (if available outside the body via threading through the trocar).

It is important to note that many of the individual elements of the techniques describe with regard to the various embodiments may advantageously be combined in particular situations. For example, as shown in FIG. 5, short removal string 46 may be used in part for alignment, or even fine realignment, of layered mesh 10 for example, before some or all clips 42 have been removed. Clips 42 may be designed have different levels of holding power or other ability to secure the layers of mesh 10 so that, for example, clips 42 in the vicinity of notch 44 may be removed before control layer 14 is removed. That is, string 46 may then be used to overcome the holding power of the lower set of clips 42 during removal.

Further, the holding functions of clips 26 may be replaced or augmented by various additions notches, similar to notch 44, but not shown in this figure, to allow contact and adhesion between mesh layer 12 and control layer 16 for example only at corners as suggested by one or more corner notches 50 as illustrated by diagonal dotted line 50. One or more separation zones, not shown in this figure but similar to zone 32 discussed above with regard to FIG. 3, may be used to separate control layer 14 in more than 2 pieces.

Such separation zones may also of separately or used to remove notches 50 and/or to remove portions of layered mesh 10, for example in corners or other locations where mesh 12 is intentionally adhered to control layer 14 by variations in the adhesion control properties of layer 14 in different areas.

As a result, some primary advantages of the techniques disclosed or discussed herein, separately or as portions of various techniques of these embodiments may be combined, may be achieved by:

causing layered mesh 10 to have a substantially lower adhesion, if any, to target area 16 than would be provided by the self-adhesive medical mesh 12 after adhesion control layer 14 was removed so that it was easier for the surgeon to deploy, temporarily align and/realign self-adhesive medical mesh 12 with target area 16, and

providing convenient techniques for the surgeon once final alignment has been achieved and is being maintained to remove control layer 14 from layered mesh 10 by one or more of the following techniques

fully or partially disengaging mechanical or adhesion mechanisms holding layers 12 and 14 together, and/or

maintaining alignment by partial adhesion between layers 12 and 14 by, for example using grabber point press at one of more location and/or using connections to layered mesh 10 such as strings,

removing some or all of the adhesion control properties of layer 14 by providing, for example, some combination of:

separating layer 14 into multiple pieces and/or

removing some or all portions of layer 14, and/or

causing layer 14 to dissolve or otherwise automatically be eliminated.

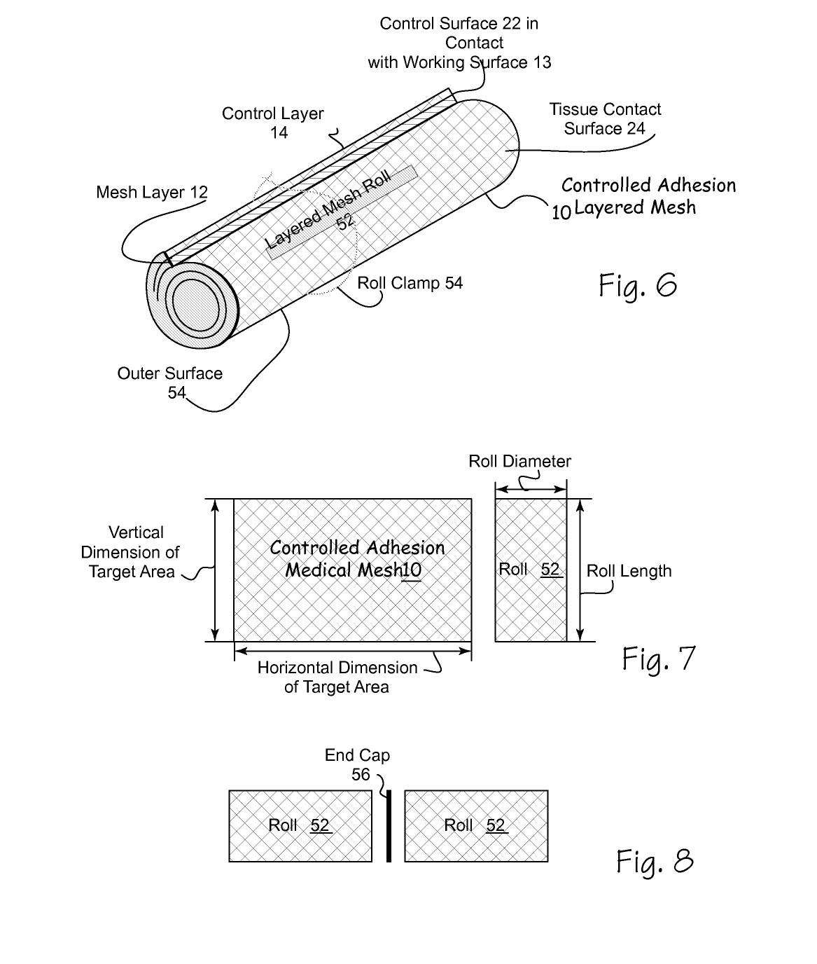

Referring now to FIG. 6, in preparation for an operation using controlled adhesion, layered mesh 10, control layer 14 and self-adhesive medical mesh layer 12 may be held together by clamps, partial adhesion areas or any other convenient means, so that control surface 22 of control layer 14 is in contact with working surface 13 of mesh layer 12. The surgeon would select or cut a section of controlled adhesion mesh 10 large enough to cover target area 16, e.g., large enough cover both the damaged area such as hernia 18 and an area of undamaged tissue preferably extended beyond the damage area in all directions. When layered mesh 10 is to be inserted in the patient's body via trocar, the surgeon may select, or fabricate, a suitable sized roll of layered mesh 10, such as rolled package 52 of controlled adhesion, layered self-adhesive medical mesh 10. String and clips are not shown for clarity.

The outer surface of roll 52 is outer surface 54 of control layer 14 which faces away from self-adhesive medical mesh layer 12 while layered mesh 10 is in an unrolled configuration. Because this surface can be controlled during fabrication, it can be made to be easily handled and grasped by the surgeon as well as having the required low or zero adhesion to the surface of mesh 12.

After preparation of layered mesh roll 52, roll 52 may be inserted into a trocar and transferred into the patient's body where the suture or other roll clamp 54 is released. Depending on the physical memory of control layer 14, layered mesh roll 52 may then partially or fully unroll within the patient's body. It may be advantageous to include a layer of plastic or a similar material, in the fabrication of control layer 14, which when allowed to unfurl after being rolled causes controlled adhesion self-adhesive medical mesh 10 to automatically fully or at least partially unfurl to a relatively planar shape for the surgeon's convenience.

Further, in one embodiment, a series of rolls 52 may be prepared and packaged by a manufacturer, held together for example by a simple suture, such as clamp 54, and be made available it the surgeon in a wide variety of sizes. Prepacked rolls 52 could made available in a limited range of rolled lengths and diameters, perhaps range selected to cover expected requirements of particular types of laparoscopic surgeries and then an appropriately size roll could be trimmed to a better shape and size.

Referring now to FIG. 7, the maximum diameter of the roll is controlled by the internal diameter of the trocar to be used and can easily be trimmed by the surgeon because outer surface 54 of control layer 14 is not going to adhere to the surgeon's gloves the way conventional self-adhesive medical mesh does. The length of roll 52 may also easily be trimmed by the surgeon because of the non-adhesion qualities of surface 54.

The surgeon can easily cut off a portion of the roll length to very quickly adjust the resultant layered mesh 10 when unfurled for the vertical dimension of the target area. Similarly, the surgeon can unroll and cut off a portion of layered mesh 10 from roll 52 to reduce the diameter of roll 52 to accommodate the horizontal dimension of the target area.

Referring now to FIG. 8, one or more end caps which may have limited or no adhesion to controlled adhesion, layered self-adhesive medical mesh 10 may be supplied with prepacked rolls 52. In one embodiment, for example including a pair of rolls 52 separated by an end cap 56, the pair of layered mesh rolls may be loaded together to reduce the surgeon time and therefor costs of the operation, for example for laparoscopic surgery for a double hernia.

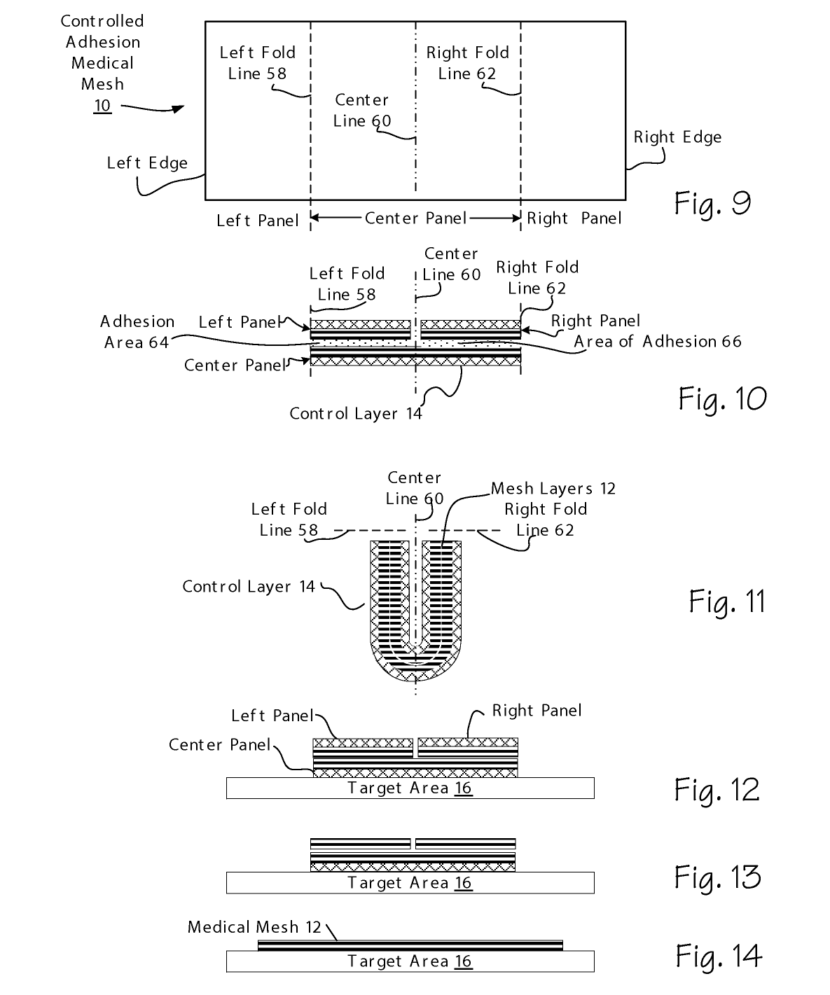

Referring now to FIGS. 9-11, control layer 14 may be applied to less than all of a first side of self-adhesive medical mesh layer 12 and/or also to the second side to provide a folded controlled adhesion self-adhesive medical mesh 10 that may be easier to deploy and use. For convenience, the surface area between left fold line 58 and right fold line 62 may be referred to as a center panel. As shown, the left panel refers to the surface area beyond left fold line 58 and the right panel refers to the surface area beyond right fold line 62.

In one embodiment, after selecting and/or preparing a piece of controlled adhesion self-adhesive medical mesh 10 sized, for example, for use with the desired size for use with a particular target area 16 size, layered mesh 10 may be folded along fold lines 58 and 62 as shown in the top view in FIG. 9.

As shown the exploded view shown in FIG. 10, folded layered mesh 10 includes a central panel with control layer 14 on the bottom and mesh 12 on the top, as well as the right and left panels with their outside edges adjacent each other in the vicinity of center line 60. As a result the mesh areas of the left and right panels separately adhere to the mesh of the central panel along adhesion areas 64 and 66.

One of the portions of control layer 14 in the left and right panels may be removed before further processing if the surgeon prefers to use the stickiness of the portions of mesh layer 12 for aid in alignment while positioning layered mesh 12. If both such layers are removed, a further control layer not shown in this figure, perhaps with different adhesion control or other properties may be positioned between the mesh 12 surface areas of the left and right panels when layered mesh 10 is further folded.

As shown in FIG. 11, when further folded down around center line 60 like a taco, layered mesh 10 may be inserted into a trocar or further reduced in size as necessary to insert via a trocar into patient's body.

As shown in FIG. 12, layered mesh 10 may be unfurled by the surgeon and/or as a result of the unfolding of adhesion control layer 14 after insertion. The surgeon may position layered mesh 10 by moving the upper surface. Layered mesh 10 when folded as shown may easily be positioned by the surgeon because the lowest surface is adhesion control layer 14. It is important to note the projection of the surface area of layered mesh 10, when folded as shown in FIG. 12, has been reduced which may be advantageous during placement.

Referring now to FIG. 13, the portion of layer 14 on the central panel may be removed by strings or the techniques as discussed above once a satisfactory temporary alignment has been achieved. Left and/or right panels of layer 14 may be removed and thereafter, mesh 12 portion of these layers must by separated by the surgeon from their adhesive contact with the mesh 12 portion of the central panel to which they've adhered. This step is much easier to perform because separating each of the subpanels from the central panel is being advantageously performed after the central has been positioned and secured to the target area.

Referring now to FIG. 14, layered mesh 10 is thereby deconstructed into the self-adhesive medical mesh 12 layer as shown in FIG. 14 and may, for example, serve as an architectural support across a hernia improved overtime by accretion of biological material. That is, time and costs for surgeries using mesh can be substantially reduced by:

i. controlling the adhesion qualities of the mesh 12 to be introduced into the body,

ii. using mesh 12 as one layer of controlled adhesion self-adhesive medical mesh 10 which can be handled, furled, unfurled, positioned easily without the problems caused by the high adhesion of mesh 12 to organic and other materials, and thereafter,

removing adhesion control layer 14 after the surgeon has determined that mesh 12 has been suitable positioned and pushed into strong contact with the organic materials within contact area 14, including the defect to be strengthened by mesh 12 and surrounding undamaged tissue.

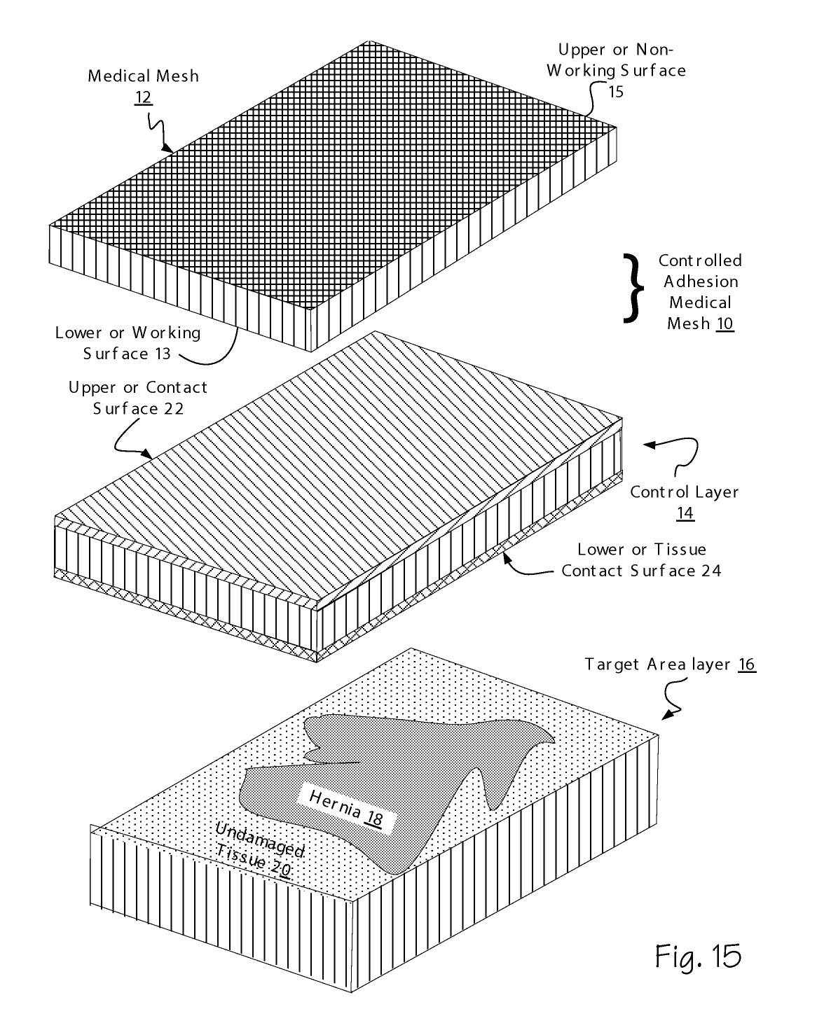

Referring now to FIG. 15, an exploded view of self-adhesive medical mesh 10 is shown position for convenience in the area of target area 16. When self-adhesive medical mesh 10 has been unfurled, unfolded or otherwise deployed within the body on target area 16, in any of the embodiments discussed and in various combinations thereof, the next step may typically be to remove control adhesion control layer 14 from controlled self-adhesive medical mesh 10, leaving the adhesive working surface, lower surface 13 of mesh 12 in contact with hernia 18 (or other area to be strengthened or repaired) and undamaged tissue 20 of target area 16.

To facilitate the deployment of mesh 10 into the body, proper placement thereof with regard to target area 16 and the subsequent removal of control layer 14 from layered mesh 10 to place mesh 12 into contact with target 16, it is important to properly control the adhesive qualities of the upper and lower surfaces 22 and 24 of control layer 14. It may be possible to select the core material of control layer 14 to wholly or partially provide the required adhesive qualities directly. Alternately, these surfaces may be formed by adding or treating surface layers of control layer 14 as shown in this figure.

When unexploded, lower surface 13 of mesh 12 may be in contact with upper surface layer 22 of control layer 14. Similarly, lower surface layer 24 of control layer 14 may be in contact with the upper surface of target area 16 including hernia 18 and surrounding undamaged tissue 20.

In a simple embodiment, control layer 14 might be a plastic material with little or no adhesion forces between lower surface 13 and upper surface 22 and similarly between lower surface 24 and target area 16. In this embodiment, some mechanism must be used to keep self-adhesive medical mesh 12 in contact with control layer 14, such as clips 26 discussed above, in order to deploy controlled adhesion self-adhesive medical mesh 10. Further, once the clips or other devices were removed, there would be little or no adhesion between control layer 14 and target area 16 that would inhibit remove of layer 14 after layered mesh 10 was properly positioned.

In some circumstances, however, the positioning of layered mesh 10 with regard to target are 16 may be advantageously improved by having at least some relatively low level of adhesion forces between lower surface 24 and target 16 as in aid in positioning. As noted above, the high level of adhesion between mesh 12 and target 16 (once in contact with each other) is one of the bases of the benefits of the medical procedure. It may be advantageous to control the level of adhesion between layer 14 and target area 16 so that mesh 10 may be movable about the surface of target area 16 for proper positioning and thereafter have sufficient adhesion to maintain that position while preparing for the next stage of the procedure, such as removing layer 14 from mesh 10.

In particular, the level of adhesion between mesh 12 and upper surface 22 should typically be greater than the adhesion between surface 24 and target 16 so that forces applied to mesh 10, after being positioned with respect to target 16, do not cause mesh 12 to unintentionally be dislocated from layer 22 and therefore from the proper position relative to target 16.

On the other hand, the total adhesion between layer 14 and layers 12 and 16 must be overcome to remove layer 14, in whole in parts, so that mesh 12 can adhere to target area 16.

The desired or appropriate levels of adhesion between layer 14 and either or both of self-adhesive medical mesh layer 12 and the surface of target 16 may also be affected by the circumstances of the surgery. For example, surgery requiring a very difficult placement of layered mesh 10 with regard to target area 16, and/or surgery in which the structure of the area to be strengthened or supported, such as hernia 18 and/or the undamaged tissue 20, may require a different selection of the adhesion strengths of layers 22 and/or 24.

One convenient material for layer 14 may be sterile paper fit for human use and temporary deployment within the human body during surgery. Such paper may be a sterile version of the paper used to protect the adhesive ends of bandages. As noted above, the reduced adhesion qualities of the paper used for layer 14 may be inherent to the material selected and/or provided by additional layers, such as upper and lower surface layers 22 and 24.

In some embodiments, upper or non-working surface 15 of mesh 12 may include a similar paper or other material layer having controlled adhesion (not shown in this figure) used to control the sterility of self-adhesive medical mesh 12 before and/or after joining mesh 12 to layer 14 to form layer 10.

In some embodiments, such as the embodiments discussed in FIG. 3 and on which use one or more tear and removal strings, layer 14 should have sufficient strength to avoid tearing, except where intended upon activation of tear string 30, while being removed by activation of one or more removal strings 36, 38 and 40 as shown for example in FIG. 3. Other combinations of adhesive strengths, the difference between the lower resistance to tearing along separation zone 32 and the higher resistance at other aspects of control layer 14, as well as the materials used, may be useful.

Similarly, for example in the embodiment(s) shown in FIGS. 9-14, using foldable panels similar configurations, it may be desirable to use materials for layer 14 having mechanical memory or recoil properties so that, when being deployed, layer 14 may tend to try to resume its unfolded state and thereby at least partially self-deploy or unfurl.

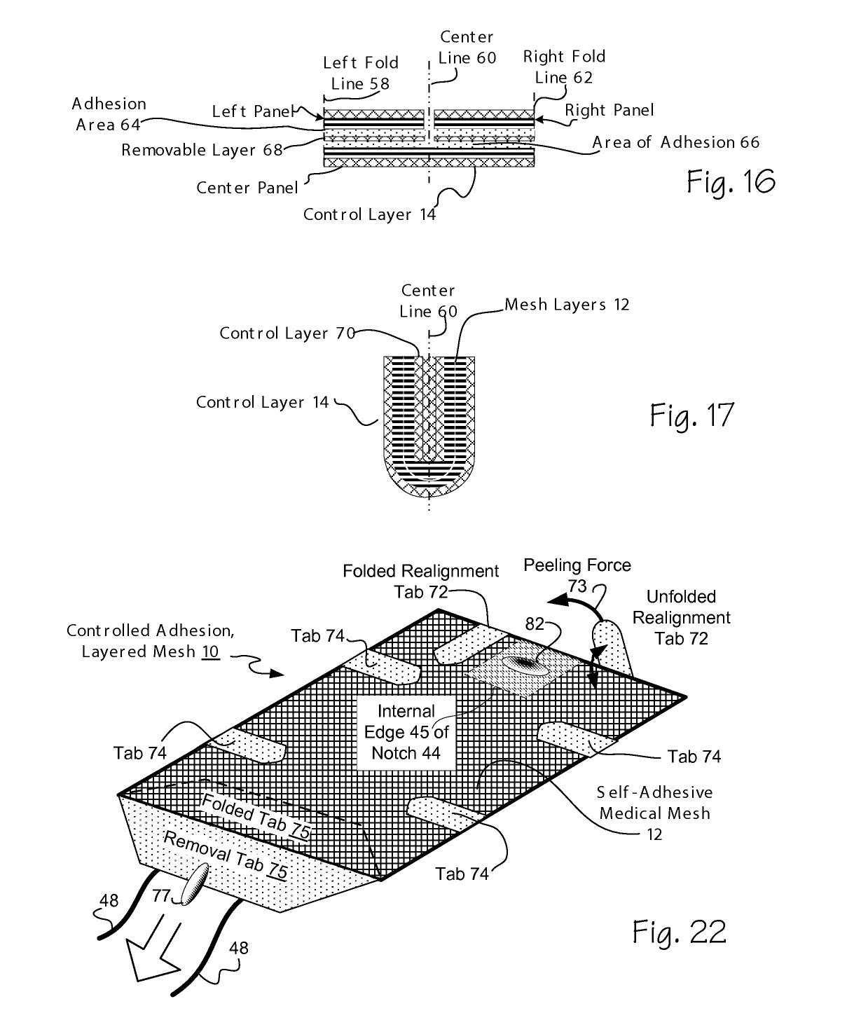

Referring now to FIG. 16, which is similar to FIG. 10 described above, controlled adhesion layer 68, with a limited level of adhesion to the folded panels of mesh 12, may be position as shown between, for example between adhesion layers 64 of the left and center panels of mesh 12 to aid in the deployment of mesh 12 at least to the configuration shown in FIG. 13.

Referring now to FIG. 17, which is similar to FIG. 11, this configuration of mesh 10 could be manufactured using a single layer of layer 14, indicated in this figure as control layer 70. Layer 70 may conveniently be thinner than layer 14 but is shown for clarity herein as a thicker layer.

Referring now to FIG. 18, a presently preferred embodiment of control layer or carrier 14 is shown in an isometric view with lower surface 24 visible on top. This is upside down view, compared to the other figures herein, and is provided for clarity of discussion of the reinforcement and anchor features as discussed below.

Control or carrier layer 14 includes a plurality of tabs 72 and 74 which can be used to perform the function of keeping control layer 14 in position against self-adhesive medical mesh 12 (not shown in this figure) during the endoscopic procedure to minimize attachment of self-adhesive medical mesh 12 to undesired structures such as the tissue removed from target area 16 to review hernia 18 and undamaged area 20. Carrier 14 is also useful to reposition controlled adhesion, layered mesh 10 before separation thereof to remove carrier 14 and adhere self adhering mesh 12 in the desired orientation adhered to target area 16. When self-adhesive mesh 12 (which would be positioned below control layer 14 in this orientation), is combined with carrier 14 to form controlled adhesion, layered mesh 10, tabs 72 and 74 would be folded over, and/or otherwise disposed on the exposed surface of mesh 12 to aid in keeping layers 12 and 14 together until self adhering mesh 12 is secured to target area 16 and carrier 14 is removed.

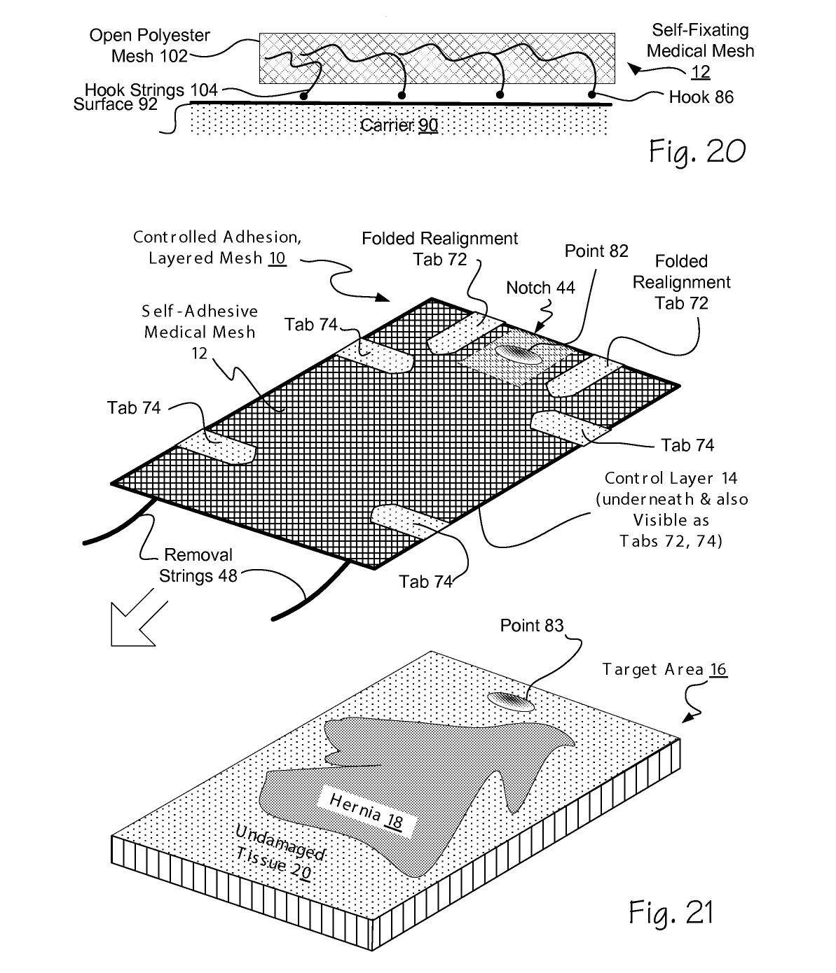

Control layer 14 may be configured from many different materials, or combination of materials, including plastic and paper. In a preferred embodiment, the material of control layer may aid in keeping these tabs folded against the working surface (the surface to contact target area 16) of self-adhesive mesh 12 as discussed in more detail in FIG. 20 below, for example by forming a crease in the material of carrier 14 at the base of each tab 72 or 74.

Notch 44, described above with regard to FIG. 5 and on, is an opening in control layer 14 which permits a small portion of the working surface of self-adhesive mesh 12 to be available during alignment of layered mesh 10 with target area 16. When the surgeon decides that layered mesh 10 has been properly aligned with target 16 and free of underlying undesired tissue or other structure, the surgeon may apply pressure on the non-working surface side of self-adhesive mesh 12 to make a limited area of adhesive contact between the working surface of mesh 12 and target area 16, for example at alignment point 82. Alignment point 82 is on the non-working surface of self adhering medical mesh 12 visible within notch 44 as illustrated below in FIG. 20. After review, if the surgeon determines that layered mesh 10 is properly aligned with target 16, the surgeon may pull removal strings 48 which may extend outside of the body via a trocar, not shown, to remove control layer 14 and finalize the position of self-adhesive mesh 12 on hernia 18 and/or undamaged tissue 20.

As described in more detail below with regard to FIG. 20, some of the tabs, such as realignment tabs 72, may serve an additional or alternate purpose when, as may occasionally happen, the surgeon may choose to realign layered mesh 10 after partial adhesive contact has been made, intentionally or not, at alignment point 82 with target are 16 or other part of the patient's body. Although described in more detail below, realignment tabs 72 are preferably configured or positioned adjacent notch 44.

Other tabs, such as holding tabs 74 which may be the same or different than realignment tabs 72, may advantageously be positioned transverse to the direction of travel of control layer 14 when being removed by operation of removal strings 48, the removal direction is indicated by an arrow. By pulling strings 48 along the plane of the working surface of self-adhesive mesh 12, the process for removing adhesive control layer 14 may thereby aid in the alignment of self-adhesive mesh 12 to the desired area.

Referring now to the composition of control layer 14, paper and similar materials useful for this purpose may be woven or made from a composition of fibers caused to stick together, such as typical paper. As illustrated below with regard for example to FIG. 15, control layer 14 has upper and lower surfaces 22 and 24 which may be part of the material of layer 14 or added coatings. Upper surface 22 must have sufficiently low adhesion to the working surface of self-adhesive medical mesh 12—and lower surface 24 must similarly have a sufficiently low adhesion to target area 16—so that pulling on removal strings along the plane of control layer 14 allows layer 14 to be removed easily, without tearing or crumpling the layer. Similarly, control layer 14 must be strong enough to resist tearing which could easily be harmful, for example, if a portion of layer 14 was torn and therefore not removed from the body before the surgical openings are closed at the conclusion of surgery.

The strength of a control layer 14 made of paper, and therefore its resistance to tearing, depends on the composition of the paper as well as the orientation of fibers to the stress lines caused by operation of removal or other handling devices such as removal strings 48. Most paper made today has a random orientation of fibers so stress lines are likely to be at the wrong angles to the randomized fibers in many places. Although making layer 14 thicker may increase its strength and resistance to tearing, thicker paper becomes more difficult to remove via trocar by using a reasonably small plurality of removal strings 48.

Although the various layers of layered mesh 10 have been shown as generally rectangular for convenience of making the figures, one simple improvement may be to make the various corners rounded and smooth, rather than square, sharp corners. Reinforcement lines 76, of some suitable material such as plastic, may be applied to a surface of control layer 13, preferably lower surface 24, to reduce the risk of tearing, crumpling or otherwise making control layer 14 more difficult to remove completely via removal strings 48.

Adding reinforcement lines 76, on the lower or upper surface of control layer 14, may increase strength of the layer and minimize stress and potential tearing or other damage. The actual layout of reinforcement lines 76—if included—may depend on many factors including the operation to be performed and the materials to be used. However, applying a pattern of reinforcement lines which radiate from the points of attachment, such as anchors 80 (shown slightly highlighted for convenience) of attachment strings 48, will substantially reduce the stress within control layer 14 when removed, for example, when strings 48 are pulled.

Further, the efficacy of reinforcement lines 76 may be enhanced by running reinforcement lines from string anchors 80 to points of high stress such as internal corners “a” and “b” of notch 44 as well as other corners of notch 44 and layer 14. Further, self-fixating medical mesh 12, such as Covidien's Progrip™ mesh may have a more complicated, 3 dimensional shape to be contoured to better lie against the area to be reinforced, such as by creating a seam in the mesh so that on portion of the mesh is intended for contact with the abdominal wall while another portion of the mesh is intended for contact with the abdominal floor. In such cases a more complicated layout of reinforcement lines 76 may be indicated by the more complicated shape of mesh 12.

Further reduction of potential tearing stress during carrier 14 removal may include rounding the corners where tabs 72 and 74 join the main body of control layer 14 and adding lines 76 along the lengths of these tabs and/or at other identified points of stress. Similarly, anchors 80 used to secure removal lines 48 to the main body of layer 14 may advantageously include circular or oval lines to transfer stress reduction from the anchors to other lines 76.

Designing a specific layout of reinforcement lines 76 for each of many different sizes and shapes of control layers 14 may be cumbersome and unnecessary, except in limited circumstances. A layout of crossed lines 76 at an angle to the direction of removal, together with reinforcement lines in the anchors and at other stress points, may be a much more convenient approach for many if not all such shapes and sizes of control layer 14.

The material used for carrier or control layer 14 may depend upon the construction of medical mesh 12. As used herein, terms self-adhesive and the like when applied medical mesh 12 are intended to describe the fact contact between the working surface of mesh 12 and target area 16 causes mesh 12 to form an adhering or bonding contact with the tissue of target area 16. For example, before the development of Covidien's so called self-fixating ProgriplM mesh, conventional mesh used in open and/or endoscopic procedures might have some level of bonding to tissue, but would typically require the addition of surgical staples and/or bio-absorbable nails and the like to hold the mesh in the proper position long enough to be secured by the growth of scar tissue after surgery.

Covidien's Progrip™ self-fixating mesh is said to be self-adhering, that is, self-adhesive to tissue in a manner analogous to the hook and loop adhering mechanism of Velcro© brand tape. That is, in addition to a medically suitable mesh which may not have sufficient stickiness on its own, if any, structure is said to be added which performs the function of hooks which adhere to tissue which forms the loop of the Velcro© like hook and loop technique. In accordance with the literature, it appears that the Progrip™ mesh is a self-gripping mesh of monofilament polyester and polylactic acid (PLA) resorbable microgrips acting as the “hooks”.

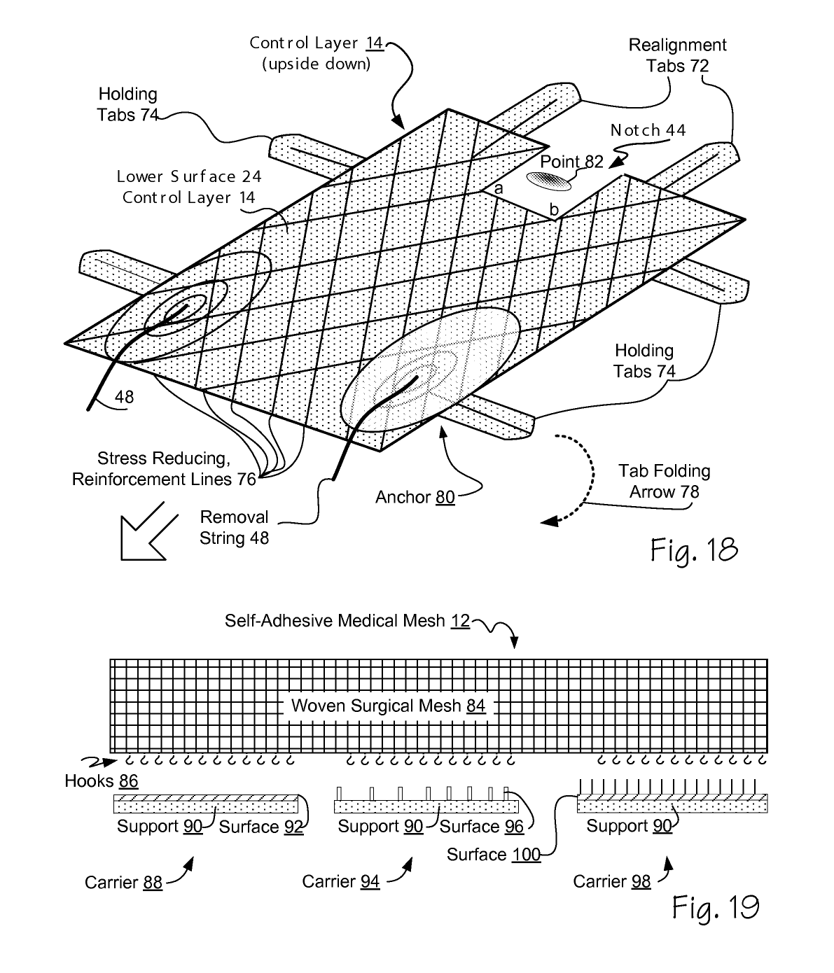

Referring now to FIG. 19, a generalized and conceptual cross sectional view of self-fixating mesh 12 is shown including non-self adhering woven medical mesh 84 and “hooks” 86 hanging thereunder on the working surface of self-fixating mesh 12 intended to be somewhat analogous to the Covidien Progrip™ self-fixating adhesion technique.

Carrier or control layer 88 represents one approach to reducing the self-adherence strength of self-adhesive medical mesh 12 to human tissue during an endoscopic insertion of layered mesh 10 through a trocar into a cavity, formed for example by balloon and maintained by inert gas, adjacent a target area and while unfurling and positioning layered mesh 10 onto target area 16. Carrier 88 includes support 90 which may be paper, plastic or similar sheet material which may include additional structural features such as notch 44, tabs 72 and 74, stress reducing lines 76, anchors 80 for removal strings 48 as well as other features discussed herein.

In this embodiment, support 90 provides the structural strength and working surface 92 is configured to not adhere to self-adhesive mesh 12 or adhere only with a sufficiently low force of adhesion so that

carrier 88 can be removed from adhesive mesh 12 without disturbing its orientation to target area 16, and

then carrier 88 can be removed from the surgical cavity without tearing, for example by trocar.

Surface 92 maybe a treated or untreated surface of support 90 and/or an additional layer such a coating applied to the surface of support 90. In the example of self-adhesive medical mesh adhering by means of a Velcro® like “hook and loop” technique, surface 92 would be configured to resist adhesion to hooks 86. For example, hooks 86 are said to become imbedded in tissue in the target area including hernia 18 and undamaged tissue 20. Surface 92 may then be sufficiently hard, impenetrable or slippery (e.g., provide a low surface tension, mechanically or chemically so as to not be penetrable by or otherwise adhere to hooks 86, that is, not able to be self-attached by hooks 86. Alternately, surface 92 may be sufficiently soft and deformable so that hooks 86 may penetrate surface 92 and yet not create substantial adhesion when carrier 88 is slid along the plane of support 90 when removed.

Referring now to carrier 94, an alternate approach is illustrated by the standoff elements of surface 96 which may, for example, protrude further from support 90 than hooks 86 protrude from the working surface of woven surgical mesh 84 so that hooks 86 cannot reach support 90 to grab hold thereof.

Still further, carrier 98 may provide a very different mechanism for reducing the self-adhesive or self-fixating quality of hooks 86. Referring back to the hook and loop adhesion of original Velcro® materials, a further development was to retain the hooks but replace the loops with fibers which had sufficiently rough surfaces, e.g., by being course or crinkled, so that the hooks would adhere to fibers which did not include loops. A non-adhering surface may be formed of smooth fibers which would not become engaged mechanically or chemically with the hooks 86.

Referring now also to FIG. 20 and continuing to refer to carriers 88, 94 and 98 of FIG. 19, self-fixating medical mesh 12 may include a non-sticky open mesh 102, e.g., of polyester, together with hook strings 104 captured by mesh 102 having at least end portions of resorbable material, e.g., of polylactic acid formed into anchors or hooks 86 which protrude therefrom. Support 90 may be formed of a sheet of polyester, or similar long chain plastic material, to which the working or adhering surface of mesh 12, e.g., hooks 86 does not adhere. Alternately, support 90 may be made of another material such, as paper, and surface 92 may be formed of polyester or a similar material suitably non-adhering for the chemical composition of the working surface of self-fixating mesh 12, i.e., non-adhering to hooks 86.

A substantial advantage of using the same or a similar material to open mesh 102 as the carrier and/or carrier surface of control layer 14 is that any concerns about tearing or other damage caused to control layer 14 may result in a remnant of that layer being left behind in the operation can be reduced or at least greatly eliminated because the material left behind accidently, if any, would be the same as the material intentionally left behind by the surgery. If fact, since hooks 86 of mesh 12 may be selected to be resorbable in the patient's body, a remnant of layer 14 which is the same, or has the same medical characteristics as mesh 12, would only reduce the force of adhesion while scar tissue is forming after the operation and after hooks make very little if any difference.

Alternately, carrier 90 may be made of a suitable low friction paper product such as the paper used to keep surgical gloves separated until use or used to wrap around Seprafilm® Adhesion Barrier membranes (Genzyme Corp, Cambridge Mass.) before and/or during surgery. Seprafilm, although useful as an adhesion barrier to reduce adhesions to polypropylene mesh in some surgeries, is itself difficult to manipulate during surgery. A paper product may be used as an envelope around the Seprafilm when handed to the surgeon and during surgery that may be useful as carrier 90.

Referring now to FIGS. 18 and 21, in one embodiment, controlled adhesion layered mesh 10 may be formed prior to insertion into the extra-abdominal cavity created cavity created for example for hernia surgery, by positioning the working surface having hooks 86 of self-adhesive medical mesh 12 onto a suitably prepared upper surface 22 of control layer 12, the lower surface 24 of which is illustrated in FIG. 18. As illustrated in FIG. 21, tabs 72 and 74 may be folded over onto the non-working surface (i.e., the surface which without hooks 86) and held in place by various techniques such as the mechanical memory of control layer 14 once folded. As highlighted in FIG. 21, a portion of the lower or working surface of mesh 12 remains unprotected within notch 44 of control layer 14.

Referring now to the surgical operation, the extra-abdominal cavity maybe be formed within layers of the appropriate portions of the abdominal wall by insertion and inflation of a balloon. The balloon may be removed and the cavity maintained during surgery by the injection of a gas. Thereafter, extensive preparation of target area 16 may be required to remove tissue and other body materials to expose the damaged body parts which may include one or more hernias 18. As noted above with regard to various figures, controlled adhesive layer 10 may be formed in various configurations suitable for the surgery to be performed and inserted within the extra-abdominal cavity.

Referring now also to FIG. 22, controlled adhesive layer 10 may then be unfolded or otherwise deployed and preliminarily positioned with regard to target area 16 so that notch 44 of control layer 14 is adjacent, and/or partially adhered, to a portion of undamaged tissue 20 on an opposite side of hernia 18 from the intended direction of removal of carrier 14, e.g., from the edge of controlled adhesion layered mesh 10 from which removal strings 48 emerge. To maintain mesh 10 in this preliminary alignment with target area 16, the surgeon may use grabbers or an other surgical instrument in the extra-abdominal cavity to apply pressure at point 82 on the non-working surface of self-fixating mesh 12 above notch 44 so that the working surface of self-fixating mesh 12 is more firmly adhered to target area 16 at point 83.

Because of the technical complexity of this type of operation, it is not uncommon for the surgeon to prefer to realign mesh 12 after preliminary alignment, for various reasons. After the portion of the working surface of mesh 12 has been pressed against point 83 in target area 16, realignment usually requires that the adherence between mesh 12 and point 83 be wholly or partially eliminated. Realignment may be more easily performed by lifting and/or peeling away, i.e., partially unfolding the folded end away of one or more of realignment tabs 72 away from non-working surface 15 of self-adhesive mesh 12 as shown in FIG. 21.

The unfolded tab or tabs may then be pulled by the surgeon using grabbers, which are likely already within the surgical cavity to gently un-attach point 82 on working surface 13 of mesh 12 from point 83 on target area 16. The direction of peeling force 73 applied by the surgeon via the grabbers is advantageous in that such force tends to lift, peel or unroll working surface 13 of mesh 12 in the vicinity of point 82 from target area 16. That is, in the case of mesh 12 using hooks 86 to adhere to target 16, only the adhesion force of hooks 86 along a line across tab 72 (where the unpeeling occurs) has to be overcome at any one time.

That is, as working surface 13 is being unpeeled, the line of hooks 86 whose adhesion is being overcome at any one time moves along surface 13, exposed to target area 16 through notch 44, from the edge of working surface 13 at the edge of control layer 14 until the adhesion forces are overcome which happens not later than when reaching the internal edge of notch 44 indicated in this figure in dotted lines. This force of adhesion to be overcome is substantially less than the force of adhesion that would have to be overcome if peeling force 73 was applied in a direction closer to parallel to the plane of controlled adhesion, layered mesh 10.

After the adhesion between points 82 and 83 has been sufficiently reduced by peeling away along peeling force 73, layered mesh 10 may be realigned and pressure re-applied by the surgeon to point 82 in order to cause the portion of mesh 12 exposed to via notch 44 to adhere to a new point 83 on target area 16 or to the same point 83 with a different alignment of layered mesh 10.

After the surgeon determines that the alignment between layered mesh 10 and target area is satisfactory, control layer 14 may be removed by operation of removal strings 44. Strings 44 preferably extend from within the patient's body to outside of the body via the trocar originally used to insert layered mesh 10 into the body. To facilitate removal of adhesion control layer 14 from layered mesh 10, any remaining folded tabs 72 and 74 may be unfolded if necessary.

As an alternate or aid to the use of removal strings 48, removal tab 75 of control layer 14 may extend beyond one end of layer 14 opposite the end of layered mesh 10 in which notch 44 is positioned. Tab 75 may be folded against non-working surface 15 of self-fixating mesh 12 indicated in dotted outline in FIG. 22 as folded tab 75 and then unfolded to the position shown in that figure as removal tab 75. Alternately, if not needed to maintain the desired juxtaposition between self-fixating mesh 12 and control layer 14 during deployment within the patient's body, tab 75 may originally be configured unfolded as shown as removal tab 75.

During removal, removal tab 75 may be grasped directly by a grabber at point 77 for removal of layer 14. Removal tab is advantageously positioned opposite adhesion point 82, and therefore may also be used during alignment and realignment, for example to more easily rotate and orient layered mesh 10. Removal strings 48 may be eliminated or advantageously used along with removal tab 75 to guide layer 14 into and along the trocar for removal.

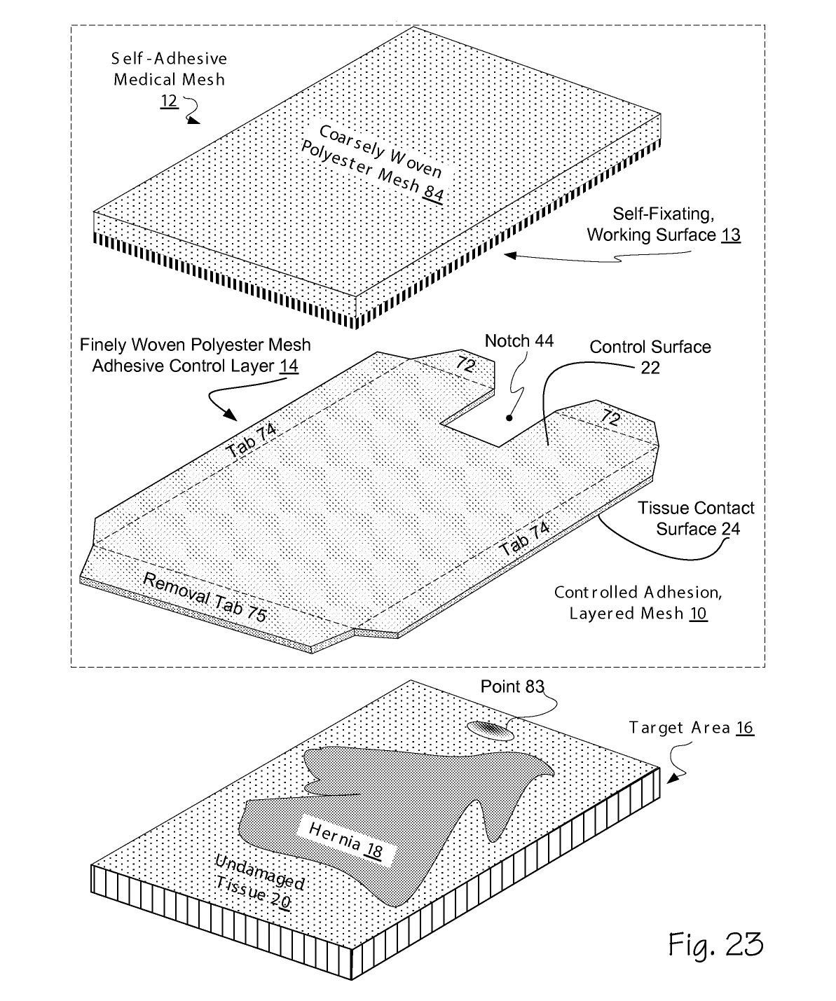

Referring now to FIG. 23, in a preferred embodiment, controlled adhesion, layered mesh 10 includes self-adhesive medical mesh 12 and adhesive control layer 14. Medical mesh 12 may be a woven mesh suitable for implanting in the human body for repairing damaged areas, such as a coarsely polyester mesh. Working surface 13 of medical mesh 12 may be self-fixating in that when placed in contact during surgery on target area 16, working surface 13 will become attached to wound 18 and undamaged tissue 20 to facilitate scar tissue growth and architectural support for the wound, such as a hernia. Working surface 13 may include hooks, anchors or other features which become imbedded in the surface area of target 16 or be naturally adhesive, or include a layer of material which by its nature adheres to target area 16.

The terms self-fixating and/or self-adhesive appear to be historical in that prior mesh did not adhere to human flesh sufficiently strongly and staples, nails or other devices including sutures were required to hold the mesh in place during and after surgery.

Adhesive control layer 14 may be a more finely woven polyester mesh than woven mesh 84 which by its density prevents hooks, anchors or other features of working surface 13 from penetrating the surface of target area 16. Alternately, layer 14 naturally, or as a result of an added layer, resists the forces of adhesion that allow mesh 12 to become adhered to target area 16.

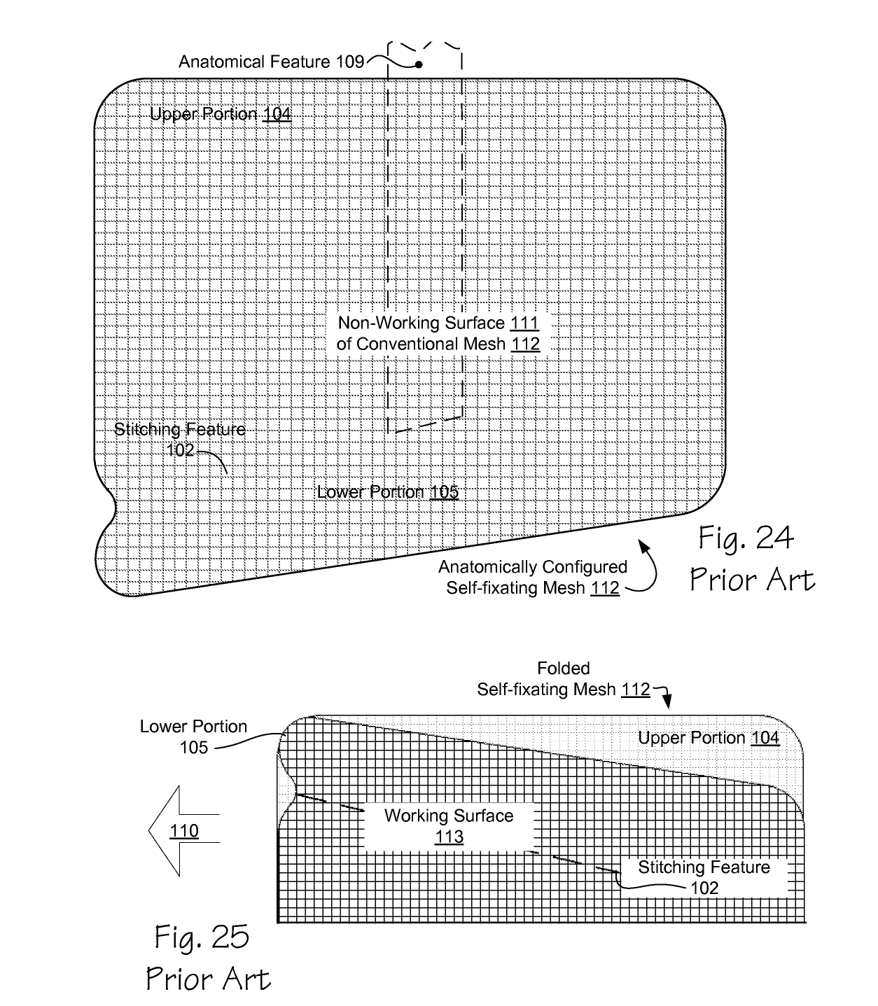

Referring now to FIG. 24, conventional self-fixating medical mesh 12 may be available in a generally rectangular configuration as shown in the figures above as well as an anatomical configuration, shown as self-fixating mesh 112 in FIG. 24. Conventional mesh 112 is partially bisected by stitching feature 102 which appears to gather a small porting of mesh 112 along stitching 102 to form a somewhat 3-dimensional form in which upper portion 104 may be positioned against the surgical patient's abdominal wall useful for covering both hernia areas 106 and 108 on either side of patient's anatomical feature 109, in case both hernias require and/or may benefit from repair and/or support. Hernias 106 and 108 and anatomical feature 109 are shown by dotted lines because in use, mesh 112 will be placed on a target area including these anatomical elements. Lower portion 105 would be positioned on the abdominal floor which extends outward from the abdominal wall in this area.

As illustrated, mesh 112 may be used for the left side of the patient and the reverse face or working surface of 113 of mesh 112 includes hooks 86 or other mechanisms for adhering mesh 112 to a target area surrounding one or both of hernias 106 and 108. The obverse surface, or non-working surface 113 of mesh 112 which is shown in this figure likely does not include mesh adhering mechanisms. If fact, at least a portion of conventional self-fixating mesh 112 on the obverse face may conventionally be treated to be less reactive to human tissue as an aid to manipulation before and during surgery.

Referring now to FIG. 25, a part of conventional mesh 112, e.g., including lower portion 105 and stitching feature 102, may be folded on top of upper portion 104 for ease of insertion via trocar in the direction of arrow 110. The cross hatched pattern of lower portion 105 is shown in black to indicate that the mesh adhering mechanism such as hooks 86 are visible in this portion of folded mesh 112.

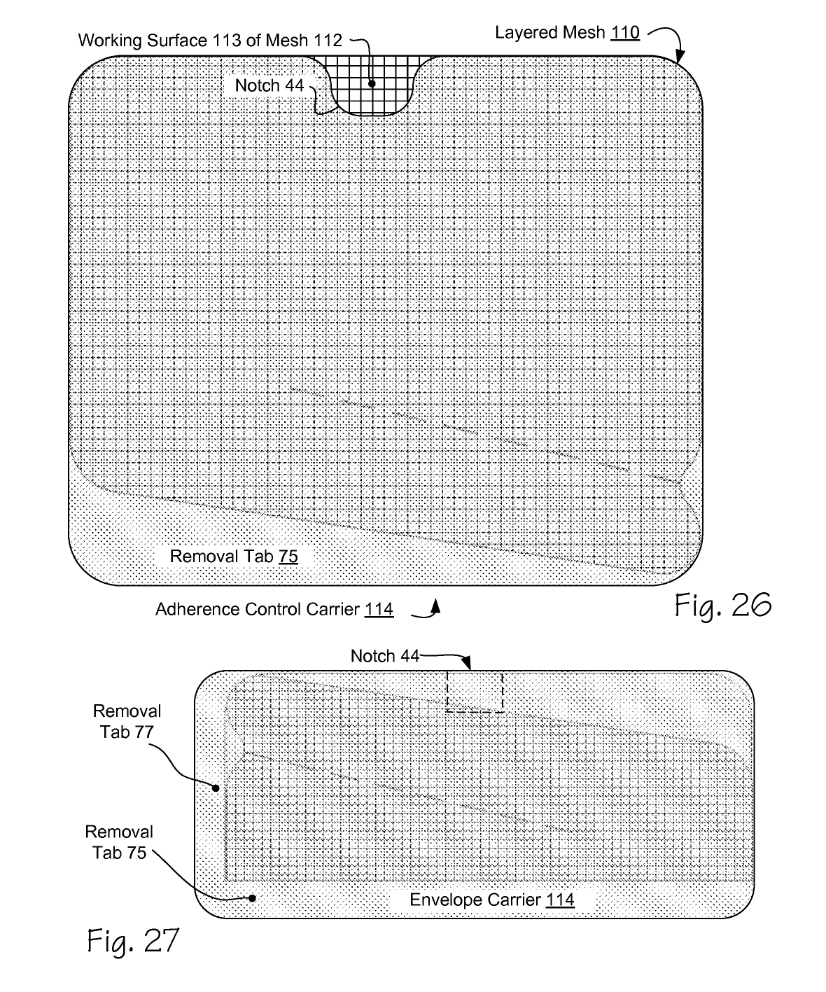

Referring now to FIG. 26, an embodiment of adhesive control layer 14 show in various previous figures is shown in this embodiment as non-fixating or non-adhesive carrier 114 and is positioned on working surface 113 of mesh 112 for form an embodiment of multi-layered self-fixating mesh 10 shown as easily positioned mesh 110. A portion of working surface 113 visible through notch 44 and thereby exposed for self-fixating against target area 16, not shown in this figure. The portion of working surface 113 not exposed via notch 44 is illustrated with reduced visibility behind carrier 114.

The portion of carrier 114 which is not covering mesh 112 may be grasped by the surgeon using grabbers or gripers for removal of carrier 114 via trocar after final placement and alignment of working surface 113 of mesh 112 on a target area during surgery and used as removal tab 75.

Referring now to FIG. 27, folded self-fixating mesh 112 shown in FIG. 25, may be positioned within an embodiment of adhesive control layer 14 shown in this figure as an envelope having a top and bottom layer and at least one open side to form layered mesh 110. One embodiment of carrier 114 is shown in this figure which is advantageously slightly larger than folded self-fixating mesh 112. Envelope 114 may include notch 44 in the reverse side of carrier 114 in this figure, the location of which is indicated by dotted lines. The open side of envelope carrier 114 is advantageously positioned at the top of the figure, as shown, along the side which included notch 44 for convenience of removal of non-adhering envelope 114. After working surface 113 visible to the target area via notch 44 has adhered to the target area in a final location and orientation, the surgeon may grasp one or more locations of envelope carrier 114, the layers of which do not enclose self-fixating mesh 112 there between for use as removal tab 75, preferably on the side of envelope carrier 114 opposite the open side including notch 44.

Alternately or additionally, another portion of envelope 114 not enclosing folded mesh 112 may also be used for removal and is marked on the figure as removal tab 77. Still further, removal tabs 75 and 77 may also be advantageously used for positioning and repositioning layered mesh 110 if necessary during surgery without risking any damage to working surface 113. However, even if a portion of envelope 114 is grasped to position or reposition layered mesh 110 which encloses mesh 112 within the area grasped, minimal interference with the self-fixating qualities of layered mesh 110 because only a minimal number of hooks 86 or other adhering mechanism will be effected.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

1. Self-attaching layered mesh for endoscopic, laparoscopic and other limited access surgery, comprising:
medical mesh having a working surface which self-attaches to tissue; and
an adhesion control layer having
i) a tissue contact surface which does not self-attach to tissue and
ii) a control surface to which the working surface does not self-attach;
said medical mesh and control layer forming a layered mesh with the working surface in contact with the control surface, said layered mesh sized for insertion during controlled access surgery via a trocar into a cavity; and
said layered mesh control layer configured to selectively self-attach to tissue in a target area to be repaired during a preliminary alignment of the medical mesh with said tissue and fully attaching thereto after removal of the control layer from the cavity via the trocar.

2. The invention of claim 1 wherein said layered mesh further comprises:
an alignment area of the working surface not blocked by the control layer,
whereby positioning the layered mesh in a desired alignment with the tissue to be repaired is not hindered by self-attachment of other areas of the working surface to tissue inconsistent with the desired alignment.

3. The invention of claim 2 wherein said layered mesh further comprises:
an alignment control area of the control layer which controls alignment of the control surface with the working surface.

4. The invention of claim 3 wherein the alignment control area further comprises:
an alignment portion of the control surface which attaches to a corresponding portion of the working surface with a lower strength of attachment than the strength of attachment between the alignment area of the working surface and the tissue so that after the layered mesh is positioned in the desired alignment with the tissue to be repaired, the control layer may be separated from the working surface for removal via trocar without loss of the desired alignment between the working surface and the tissue to be repaired.

5. The invention of claim 4 wherein the layered mesh includes a plurality of alignment control areas and the strength of attachment of the plurality of alignment control areas to the tissue to be repaired is less than the strength of attachment between the alignment area of the working surface and the tissue.

6. The invention of claim 3 wherein the alignment control area further comprises:
an extension of the control layer extending across a first edge of the working surface to the non-working surface forming a barrier against movement of the mesh within the layered mesh in at least one direction.

7. The invention of claim 6 wherein the alignment control area further comprises:
a plurality of extensions of the control layer extending across a plurality of edges of the working surface to the non-working surface forming a barrier against movement of the mesh in a plurality of directions.

8. The invention of claim 6 wherein the alignment area of the working surface is adjacent an alignment edge of the working surface and the alignment control area further comprises:
a plurality of extensions of the control layer extending across a plurality of edges of the working surface to the non-working surface forming a barrier against movement of the mesh in a plurality of directions, at least one of said plurality of extensions extending across said alignment edge.

9. The invention of claim 8 wherein said at least one of said pluralities of extension of the control layer extending across said alignment edge further comprises:
a tab in contact with the non-working surface of the mesh, said tab easily liftable from the non-working surface during surgery to aid in peeling the alignment area of the working surface from tissue in the cavity during realignment of the layered mesh.

10. The invention of claim 8 wherein at least one of the plurality of extensions further comprises:
a removal tab adjacent an edge of the control layer opposite the alignment area, liftable from the non-working surface during surgery to aid in removing the control layer from the cavity.

11. The invention of claim 6, wherein the extension of the control layer extends across the non-working surface to a second edge thereof to form a barrier against movement of the mesh within the layered mesh in the direction of the first and in the direction of the second edge.

12. The invention of claim 11 wherein the control layer does not form a barrier against movement of the mesh within the layered mesh in the direction of a third edge of the mesh and wherein the control layer further comprises:
a removal tab grippable by an instrument in the cavity for removal of the control layer from the layered mesh in the cavity in the direction of the third edge.

13. The invention of claim 1 where in the control layer further comprises:
lines of reinforcement material resisting tearing forces applied to the control layer during endoscopic removal of the control layer.

14. The invention of claim 1 wherein the mesh is a woven surgical material one surface of which is configured as the working surface, wherein the control layer further comprises:
a second mesh of surgical material woven substantially more tightly than the mesh to reduce or eliminate self-attachment thereto by the working surface.

15. The invention of claim 14 wherein the control layer further comprises:
a layer of non-woven surgical material applied to one surface of the second mesh to further reduce or to eliminate self-attachment thereto by the working surface.

16. An adhesion control carrier for use with self attaching medical mesh in laparoscopic and other limited access surgery, comprising:
an adhesion control layer having a tissue contact surface which does not self-attach to tissue and a control surface to which a self-attaching surface of the medical mesh does not self-attach, the control layer configured to selectively self-attach to tissue in a target area to be repaired during a preliminary alignment of the medical mesh with said tissue and fully attaching thereto after removal of the control layer from the cavity via the trocar.
